(19) United States
(12) Patent Application Publication
(10) Pub. No.: US 2024/0091730 A1
(43) Pub. Date: Mar. 21, 2024

(54) METHOD FOR STABILIZING EFFECTIVE INGREDIENT BY USING MINERAL MATERIAL

(71) Applicants: LG HOUSEHOLD & HEALTH CARE LTD., Seoul (KR); IUCF-HYU (INDUSTRY-UNIVERSITY COOPERATION FOUNDATION HANYANG UNIVERSITY), Seoul (KR)

(72) Inventors: Jun Seok YEOM, Seoul (KR); Eun Chul CHO, Seoul (KR); Ji Won LIM, Seoul (KR); Hyo Jin BONG, Seoul (KR); Seon A JEONG, Seoul (KR); No Jin PARK, Seoul (KR); Woo Sun SHIM, Seoul (KR)

(73) Assignees: LG HOUSEHOLD & HEALTH CARE LTD., Seoul (KR); IUCF-HYU (INDUSTRY-UNIVERSITY COOPERATION FOUNDATION HANYANG UNIVERSITY), Seoul (KR)

(21) Appl. No.: 18/270,116

(22) PCT Filed: Dec. 28, 2021
(86) PCT No.: PCT/KR2021/095141

§ 371 Date: Jun. 28, 2023

(30) Foreign Application Priority Data
Dec. 29, 2020 (KR) 10-2020-0186127
Jun. 29, 2021 (KR) 10-2021-0084969

(51) Int. Cl. B01J 13/20; C11D 1/62; C11D 3/12; C11D 3/50; C11D 17/00

(52) U.S. Cl. CPC B01J 13/206; C11D 1/62; C11D 3/1213; C11D 3/50; C11D 17/0039

(57) ABSTRACT

The present invention relates to the stabilization of an effective ingredient by using a mineral material. In the present invention, the effective ingredient can be stably supported using the mineral material, and a microcapsule obtained by the manufacturing method according to the present invention, when discharged to nature, causes no environmental problems due to encapsulation ingredients thereof being the same as soil ingredients, and thus can avoid micro-plastic issues.

### TECHNICAL FIELD

The present invention relates to a method for stabilizing an active ingredient using a mineral material. More specifically, the present invention relates to a technology for stabilizing an active ingredient that can escape from microplastic issues, and relates to a technology that can stably load an active ingredient using a mineral material rather than a microplastic.

### BACKGROUND ART

Encapsulation is a general term for the form of collecting an active ingredient in a material corresponding to an outer wall for effective delivery of the active ingredient. Such a capsule can reduce side effects, maintain the stability of the active ingredient, and reduce cost issues by accurately delivering the active ingredient according to the purpose even with the use of a small content of the active ingredient. Through this, the capsules are applied for various forms and purposes in industrial fields, such as targeted delivery of drugs in the medical/pharmaceutical field, maintaining the stability of fragrances in sanitary products and diffusing perfume at the time users desire, maintaining the stability of an efficacious substance against external stimuli such as light, heat and the like in the cosmetic field, and maintaining the stability of insecticides and nutritional components in the agricultural and food fields and enhancing the absorption efficiency thereof.

However, international organizations are pointing out microcapsules as one of the causes of microplastics (small plastic pieces with a diameter of 5 mm or less) pollution that can accumulate without degradation in nature and cause serious environmental pollution. According to the ECHA report (2019), materials designed to be immediately degradable called readily biodegradable materials were selected as materials that can escape the microplastic issue when inorganic or biodegradable polymer materials are used as they are. However, capsules made of inorganic materials such as silica, a material that is not in the category of microplastics, have a problem in that they break easily due to weakness in tension or impact generated during drying. In addition, when capsules are prepared using natural polymers, there is a problem in that the stability of the capsules is not maintained because the active ingredients inside the capsules are eluted due to the inherent microporosity of the material. When crosslinking is used to increase stability by reducing microporosity, there is a problem in that degradability is lowered and biodegradability is not exhibited. In addition, when a substance in which an ester bond is introduced so that it is easily degraded is used, it does not withstand the harsh conditions of the formulation (pH change, temperature change) and thus is degraded before use, but to this day no solution has been found and there may be no solution.

Silica, an inorganic material, is a safe material that does not affect living bodies and is widely used in pharmaceuticals, cosmetics, and daily supplies. Silica is a safe material because it occupies most of a soil component, which is a material constituting the land, and is a material constituting a cell wall in the case of diatoms, which are microorganisms. Therefore, studies on encapsulation of an active ingredient using silica are in the spotlight, but as mentioned above, since silica is easily broken by tension generated during drying, unlike polymers, research to compensate for this is needed.

### RELATED ART DOCUMENT

### Non-Patent Document 1: ANNEX XV RESTRICTION REPORT, PROPOSAL FOR A RESTRICTION: SUBSTANCE NAME(S): intentionally added microplastics, DATE: 22 Aug. 2019.

### DISCLOSURE

### Technical Problem

As a result of research to develop microcapsules using mineral materials, which are inorganic materials, the present inventors completed the present invention by confirming that when microcapsules comprising an active ingredient are prepared by mixing a continuous phase comprising an emulsifier with a dispersed phase comprising an encapsulation component and an active ingredient and then curing the encapsulation component, since the encapsulation component is the same as soil components, there is no environmental problem when the encapsulation component is discharged into nature.

Accordingly, the present invention is directed to providing a method of preparing microcapsules having high versatility, compatibility with nature, and stability, microcapsules prepared by the method, and a laundry product comprising the microcapsules.

### Technical Solution

The present invention provides a method of preparing microcapsules, comprising: preparing an emulsion by mixing a continuous phase containing an emulsifier and a dispersed phase containing an encapsulation component and an active ingredient: and encapsulating the emulsion, wherein the emulsifier comprises one or more selected from the group consisting of an anionic surfactant, a cationic surfactant, an amphoteric surfactant, and a nonionic surfactant, and the encapsulation component comprises one or more selected from the group consisting of a silica precursor, a titanium oxide precursor, and a zirconium oxide precursor.

In addition, the present invention provides microcapsules prepared by the above-described method of preparing microcapsules and having a particle size of 0.1 to 1,000 μm.

In addition, the present invention provides a laundry product comprising the above-described microcapsules.

In addition, the present invention provides use of microcapsules prepared by the above-described method of preparing microcapsules and having a particle size of 0.1 to 1,000 μm for the manufacture of a laundry product.

### Advantageous Effects

The present invention provides a method for stabilizing an active ingredient using a mineral material.

In the present invention, the active ingredient can be stably loaded by using the mineral materials, and since microcapsules prepared by the preparation method according to the present invention have the same encapsulation components as soil components, there is no environmental problem when the encapsulation component is discharged into nature, and thus microplastic issues can be avoided. In addition, since the microcapsules prepared by the preparation method according to the present invention have an outer wall of a capsule having a dense structure, elution of the active ingredient can be prevented.

### DESCRIPTION OF DRAWINGS

FIG. 1 is a schematic diagram of a method of preparing a microcapsule according to the present invention.

FIG. 2 shows the results of confirming the shape of microcapsules prepared in examples of the present invention using scanning electron microscope (SEM). Specifically, FIG. 2A is an SEM picture of Example 8, FIG. 2B is an enlarged picture of Example 8, FIG. 2C is a picture of a fracture surface of Example 8, and FIG. 2D is a picture of the fracture surface of Example 40.

FIG. 3 shows the result of confirming a particle size of microcapsules prepared in Example 8 of the present invention using the Mastersizer 3000.

FIG. 4 shows the results of confirming a preservation amount of the fragrance in capsules over time in an environment of 50° C. in the presence of an emulsifier for microcapsules prepared in Examples 8 and 40 of the present invention.

### MODES OF THE INVENTION

The present invention relates to a method of preparing microcapsules, comprising: preparing an emulsion by mixing a continuous phase containing an emulsifier and a dispersed phase containing an encapsulation component and an active ingredient: and encapsulating the emulsion, wherein the emulsifier comprises one or more selected from the group consisting of an anionic surfactant, a cationic surfactant, an amphoteric surfactant, and a nonionic surfactant, and the encapsulation component comprises one or more selected from the group consisting of a silica precursor, a titanium oxide precursor, and a zirconium oxide precursor.

Hereinafter, the configuration of the present invention will be described in detail.

In the present invention, microcapsules refer to microscopic particles surrounding liquid, solid or paste-type active ingredients, and may have a particle size of 0.1 to 1,000 μm. The microcapsules can physically and chemically protect the active ingredient from an external environment, and also control the release of the active ingredient.

In the present invention, the microcapsules can be prepared through the following: preparing an emulsion by mixing a continuous phase containing an emulsifier and a dispersed phase containing an encapsulation component and an active ingredient (hereinafter, emulsion preparation): and encapsulating the emulsion (hereinafter, encapsulation).

In the present invention, the emulsion preparation may be a step of mixing the continuous phase and the dispersed phase to prepare the emulsion.

In the present invention, the continuous phase is an aqueous phase and may comprise an emulsifier.

In one embodiment, any solvent commonly used in the art may be used as a solvent of the continuous phase, and specifically, distilled water may be used.

In one embodiment, the emulsifier is a material used as a backbone so that the encapsulation component to be described below can be converted into the outer wall of the capsule. Emulsifiers commonly used in the art may be used as such an emulsifier, and specifically, one or more selected from the group consisting of an anionic surfactant, a cationic surfactant, an amphoteric surfactant, and a nonionic surfactant may be used.

The cationic surfactant may comprise one or more selected from the group consisting of a trimethylalkylammonium salt, a dialkyldimethylammonium salt, and an alkylbenzylmethylammonium salt, the anionic surfactant may comprise one or more selected from the group consisting of fatty acid sodium, a monoalkyl sulfate, an alkylpolyoxyethylene sulfate, an alkylbenzenesulfonate, and a monoalkylphosphate, the amphoteric surfactant may comprise one or more selected from the group consisting of an alkylsulfobetaine and an alkylcarboxybetaine, and the nonionic surfactant may comprise one or more selected from the group consisting of a fatty alcohol, a polyoxyethylene alkyl ether, a fatty acid sorbitan ester, a fatty acid diethanolamine, and an alkyl monoglyceryl ether.

Preferably, the emulsifier may be the cationic surfactant, and specifically, cetyltrimethylammonium bromide (CTAB) or cetyltrimethylammonium chloride (CTAC) may be used. Since hydrolysis and reaction at the interface of the CTAB or CTAC are promoted not only by the emulsification effect but also by the amine group present in the structure, a more uniform reaction may be induced and dense microcapsules may be prepared.

In one embodiment, a content of the emulsifier is not particularly limited, and may be 0.00001 to 10 parts by weight, 0.0001 to 10 parts by weight, 0.0002 to 5 parts by weight, 0.0005 to 2 parts by weight, or 0.002 to 1.2 parts by weight, based on a total weight (100 parts by weight) of the emulsion. When a content of the emulsifier is less than 0.00001 parts by weight, there is a problem that no emulsification occurs, and when it exceeds 10 parts by weight, there is a risk that the active ingredient in the capsule may be eluted due to excessive components of the emulsifier.

In the present invention, the dispersed phase is an oil phase and comprises an encapsulation component and an active ingredient.

In one embodiment, a solvent of the dispersed phase is not particularly limited as long as it is a solvent that is immiscible with the continuous phase. When distilled water is used as the solvent of the continuous phase, one or more selected from the group consisting of a hydrocarbon-based solvent: a solvent comprising an ether group: a solvent comprising an ester group: a solvent comprising a ketone group: a solvent comprising a benzene; a haloalkane-based solvent: and a silicone-based solvent may be used as the solvent of the dispersed phase.

The hydrocarbon-based solvent may be selected from compounds with a linear or non-linear structure such as pentane, hexane, cyclohexane, heptane, octane, isododecane, and dodecane, the solvent comprising the ether group may be selected from ethyl ether, butyl ether, and methyl-t-butyl ether, and the solvent comprising the ester group may be selected from ethyl acetate, butyl acetate, and ethyl butyrate. In addition, the solvent comprising the ketone group may be methyl ethyl ketone, the solvent comprising benzene may be selected from benzene, toluene, and xylene, the haloalkane-based solvent may be selected from dichloromethane, dichloroethane, chloroform, and carbon tetrachloride, and the silicone-based solvent may be selected from dimethicone and cyclomethicone.

In the present invention, the encapsulation component is a component constituting the outer wall of the capsule and can be converted into a mineral material during the encapsulation. The encapsulation may be hydrolysis.

In one embodiment, a mineral material precursor may be used as the encapsulation component, and the mineral material precursor may be one or more selected from the group consisting of a silica precursor, a titanium oxide precursor, and a zirconium oxide precursor.

In one embodiment, the mineral material precursor may comprise one or more compounds selected from the group consisting of compounds represented by Chemical Formulas 1 to 4 below.

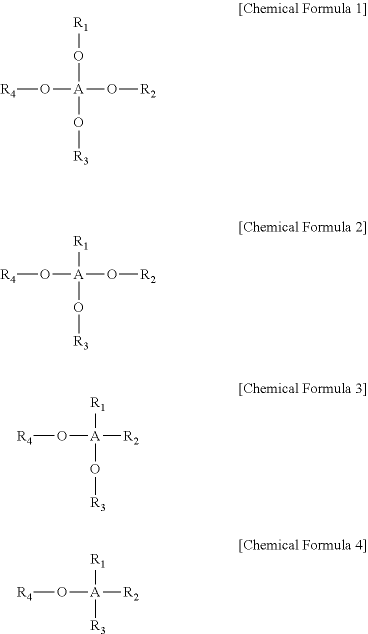

In Chemical Formulas 1 to 4, A may be silicon, titanium, or zirconium, R₁ to R₄ may each independently be hydrogen or an alkyl group having 1 to 8 carbon atoms with a functional group substituted or unsubstituted at the terminal, and the functional group may comprise amine, hydroxy, amide, carboxy, vinyl, epoxy, phenyl, or mercapto.

In one embodiment, the silica precursor may comprise one or more selected from the group consisting of tetramethyl orthosilicate, tetraethyl orthosilicate, tetrapropyl orthosilicate, tetrabutyl orthosilicate, methyltrimethoxysilicate, dimethyldimethoxysilicate, trimethylmethoxysilicate, trimethylethoxysilicate, butyltrimethoxysilicate, N-propyltrimethoxysilane, N-octyltrimethoxysilane, aminopropyltrimethoxysilicate, phenyltrimethoxysilane, 3-mercaptopropyltrimethoxysilane, and methyldimethoxysilane, the titanium oxide precursor may comprise one or more selected from the group consisting of titanium methoxide, titanium ethoxide, and titanium butoxide, and the zirconium oxide precursor may comprise one or more selected from the group consisting of zirconium methoxide, zirconium ethoxide, and zirconium butoxide.

In one embodiment, a content of the encapsulation component is not particularly limited, and may be 0.001 to 30 parts by weight, 0.01 to 25 parts by weight, or 0.1 to 20 parts by weight, based on a total weight (100 parts by weight) of the emulsion. When a content of the encapsulation component is less than 0.001 parts by weight, even when the encapsulation occurs, there is a risk that a problem may occur in which the outer wall of the capsule is formed too thin to maintain, and when the content exceeds 30 parts by weight, the distinction between the dispersed phase and the continuous phase becomes ambiguous, and there is a risk that capsules may not be formed.

In the present invention, the active ingredient is a substance whose activity is desired to be maintained by the resulting capsule, and the activity of the active ingredient can be expressed later when the outer wall of the capsule is destroyed. When the active ingredient is a liquid at room temperature, it can replace the dispersed phase as a solvent. The active ingredient may comprise one or more selected from the group consisting of a perfume, a sunscreen, a dye, a catalyst, an antioxidant, and a drug.

In one embodiment, when the active ingredient is a perfume, the present invention may provide a perfume product comprising the microcapsule. The perfume product may comprise a perfume spray, a perfume liquid product, a cleaning agent, a face wash, a body product, a hair product, or a laundry product. The laundry product is a product used to treat clothes, and may comprise, but is not limited to, a laundry detergent, a fabric softener, a perfume additive, a clothing deodorant, a dry cleaning agent, a stain remover, and a bleach.

When the active ingredient is a sunscreen, the present invention may provide a cosmetic composition for UV blocking, comprising the microcapsule. Examples of the sunscreen may comprise inorganic sunscreens such as titanium dioxide (TiO₂), zinc oxide (ZnO), silicate, or talc: and organic sunscreens such as isoamyl-p-methoxycinnamate, octylmethoxycinnamate, ethylhexyltriazone, oxybenzone, diethylaminohydroxybenzoyl hexylbenzoate, octocryleneoctylsalicylate, butylmethoxydibenzoylmethane, octylsalicylate, benzophenone, or anthranilate.

In addition, when the active ingredient is a functional substance or poorly soluble substance, the microcapsule of the present invention may be provided as a carrier for improving the stability of the functional substance or poorly soluble substance. Examples of the functional substance or poorly soluble substance may comprise antioxidants such as retinol or resverastrol, poorly soluble substances such as cholesterol or ceramide, and warm and cold sensory stimulating substances such as menthol and vanillyl butyl ether.

In one embodiment, a content of the active ingredient is not particularly limited, and may be 0.001 to 20 parts by weight, 0.01 to 15 parts by weight, or 1 to 10 parts by weight, based on a total weight (100 parts by weight) of the emulsion.

In one embodiment, a content of the encapsulation component may be 0.5 to 3 times a content of the active ingredient. When a content of the encapsulation component is more than 3 times a content of the active ingredient, there is a risk that gelation may occur due to aggregation between particles, and when the content is less than 0.5 times, there is a risk of phase separation so that it is good to keep a content ratio at 0.5 to 3 times.

In addition, in one embodiment, a content of the encapsulation component and the active ingredient may be 35 parts by weight or less based on a total weight (100 parts by weight) of the emulsion, and specifically, 10 to 35 parts by weight, 11 to 35 parts by weight, or 15 to 30 parts by weight. When a content of the encapsulation component and the active ingredient exceeds 35 parts by weight, there is a risk that it will be difficult to stabilize the active ingredient, and aggregation between particles and gelation may occur. In addition, when a content of the encapsulation component and the active ingredient is less than 10 parts by weight, there is a risk of phase separation.

In the emulsion preparation of the present invention, an emulsion may be prepared by mixing the above-described continuous phase and dispersed phase.

In one embodiment, a content of the dispersed phase may be 35 parts by weight or less, to 35 parts by weight, 11 to 35 parts by weight, or 15 to 30 parts by weight, based on a mixed weight (100 parts by weight) of the dispersed phase and the continuous phase.

In one embodiment, the emulsion preparation may be performed by adding the dispersed phase to the continuous phase and may be performed under stirring.

In one embodiment, the stirring may be performed at 1 to 16,000 rpm, 5 to 13,000 rpm, or 10 to 10,000 rpm at 20 to 30° C. or room temperature. In addition, a stirring time may vary depending on a volume of the emulsion to be prepared.

In the present invention, the encapsulation comprises preparing microcapsules by encapsulating the emulsion prepared in the above-described emulsion preparation.

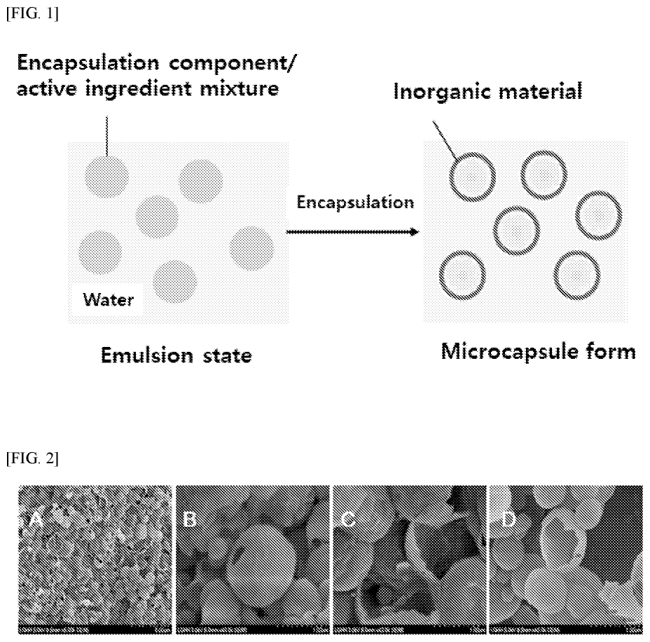

As shown in FIG. 1, stable microcapsules comprising active ingredients can be prepared through curing (or solidification) of encapsulation components. Specifically, since the encapsulation component comprises an alkoxy, it is initially mixed in a dispersed phase, which is a core material, but when hydrolysis occurs at the interface of the emulsion, the encapsulation component is changed to have hydrophilicity and is laminated at the interface. In this process, solidification occurs and a solid outer wall is formed.

In one embodiment, a time interval between the emulsion preparation and the encapsulation may be 1 hour to 14 days.

In one embodiment, the encapsulation may be performed under a weakly acidic condition. The weakly acidic condition may be a pH of 2 to 5 or a pH of 2 to 4. Under the weakly acidic condition, an outer wall of the capsule having a dense structure can be prepared. When the pH is outside the above range, desired micro-sized particles (capsules) may not be formed, and in addition, even when the particles are formed, a thickness of the outer wall is low or porous particles are formed so that there is a risk that the active ingredient is not stably loaded in the capsule and will be easily eluted.

In one embodiment, the encapsulation for preparing microcapsules may be performed at 20 to 30° C. or room temperature for 10 minutes to 48 hours. In order to promote the reaction, when the temperature is raised, micro-sized particles (capsules) are not formed, and even when the particles are formed, since there is a risk that the thickness of the outer wall is low or porous particles will form, it is preferable to adjust a reaction temperature and time as described above.

In one embodiment, the encapsulation may be performed in a state in which the emulsion is allowed to stand without stirring. Specifically, the present invention can form the outer wall of the capsule having a dense structure by performing the encapsulation in a state in which the emulsion of which the reaction is not sped up is allowed to stand.

In one embodiment, the encapsulation may be performed one or more times, specifically 2 to 3 times. A second encapsulation may be performed by adding an encapsulation component to the emulsion after the first encapsulation is completed and causing them to react. The encapsulation component used in the second encapsulation may be the same as the encapsulation component used during the first reaction, or a different type of encapsulation component may be used.

When the encapsulation is performed 2 times, microcapsules having a double wall can be prepared, and when the microcapsule is prepared with a double wall, a silica content is increased and the capsule itself is thick so that the active ingredient can be stably preserved even in harsh environments.

In addition, the present invention relates to microcapsules prepared by the above-described method of preparing microcapsules.

An average particle size of the microcapsules according to the present invention may be 0.1 to 1,000 μm, 0.1 to 100 μm, 0.5 to 80 μm, or 1 to 5 μm. When the average particle size of the microcapsules is less than 0.1 μm, a content of the active ingredient loaded in the capsules is too small so that even when the microcapsules are applied, too little a content is expressed, and there is a risk that an effect may not appear. In addition, when the average particle size exceeds 1,000 μm, there is a problem that the microcapsules are easily broken by tension generated while the capsules made of mineral materials are dried.

In addition, an outer wall thickness of the capsules may be 0.01 to 1 μm. In addition, the present invention relates to a laundry product comprising the above-described microcapsules.

In one embodiment, the laundry product may comprise a laundry detergent, a fabric softener, a perfume additive, a clothes deodorant, a dry cleaning agent, a stain remover, or a bleach.

In one embodiment, a content of microcapsules in the laundry product may be 0.5 to 20 parts by weight or 1 to 10 parts by weight based on a total weight.

In addition, the present invention relates to use of microcapsules prepared by the above-described method of preparing microcapsules and having a particle size of 0.1 to 1,000 μm for the manufacture of a laundry product.

Hereinafter, the present invention will be described in detail by way of examples. The following examples merely illustrate the present invention, and the scope of the present invention is not limited by the following examples. The examples are merely provided to complete the disclosure of the present invention and to fully convey the scope of the invention to those skilled in the art, and the invention is only defined by the scope of the claims.

### EXAMPLE

Hereinafter, raw materials used in examples, comparative examples, and experimental examples were purchased from commercial cosmetic raw material manufacturers or reagent manufacturers.

### Examples 1 to 10. Preparation of microcapsules using encapsulation components

Microcapsules were prepared according to the compositions and contents of Table 1 First, a continuous phase was prepared by dissolving an emulsifier (CTAB) in distilled below. water. At the same time, a dispersed phase was prepared by mixing an encapsulation component and a perfume well. The dispersed phase was added to the continuous phase and stirred at 8,000 rpm to prepare an emulsion, and encapsulation was performed under weakly acidic conditions.

Meanwhile, in Comparative Example 1, an emulsion in which a perfume was emulsified according to the compositions and contents of Table 1 was prepared.

First, a continuous phase was prepared by dissolving an emulsifier in distilled water. Then, a perfume which is a dispersed phase was added to the continuous phase and stirred at 8,000 rpm to prepare an emulsion.

In the present invention, except for Experimental Example 6, the prepared microcapsules were stabilized for 24 hours under constant temperature and humidity conditions of 30° C. and 50% humidity and then used.

TABLE 1

| | Compara- | | | | | | | | | | |
|---|---|---|---|---|---|---|---|---|---|---|---|
| | tive | Example | Example | Example | Example | Example | Example | Example | Example | Example | Example |
| | Example 1 | 1 | 2 | 3 | 4 | 5 | 6 | 7 | 8 | 9 | 10 |
| Titanium | | 10 | | | | | | | | | |
| methoxide | | | | | | | | | | | |
| Titanium | | | 10 | | | | | | | | |
| ethoxide | | | | | | | | | | | |
| Titanium | | | | 10 | | | | | | | |
| butoxide | | | | | | | | | | | |
| Zirconium | | | | | 10 | | | | | | |
| methoxide | | | | | | | | | | | |
| Zirconium | | | | | | 10 | | | | | |
| ethoxide | | | | | | | | | | | |
| Zirconium | | | | | | | 10 | | | | |
| butoxide | | | | | | | | | | | |
| Tetramethyl | | | | | | | | 10 | | | |
| orthosilicate | | | | | | | | | | | |
| Tetraethyl | | | | | | | | | 10 | | |
| orthosilicate | | | | | | | | | | | |
| Tetrapropyl | | | | | | | | | | 10 | |
| orthosilicate | | | | | | | | | | | |
| Tetrabutyl | | | | | | | | | | | 10 |
| orthosilicate | | | | | | | | | | | |
| Perfume | 10 | 10 | 10 | 10 | 10 | 10 | 10 | 10 | 10 | 10 | 10 |
| Cetyltrimonium | 0.05 | 0.05 | 0.05 | 0.05 | 0.05 | 0.05 | 0.05 | 0.05 | 0.05 | 0.05 | 0.05 |
| bromide (CTAB) | | | | | | | | | | | |
| Water | To 100 | To 100 | To 100 | To 100 | To 100 | To 100 | To 100 | To 100 | To 100 | To 100 | To 100 |

### Experimental Example 1. Fragrance Intensity Evaluation after Washing

Washing evaluation was performed to confirm the residual fragrance enhancing performance of the microcapsules prepared in Examples 1 to 10 and Comparative Example 1.

First, four commercially available cotton towels (30 cm×20 cm) (100% cotton, Songwol Towel Co., Ltd.) were used as test fibers. The cotton towels were washed 5 times in a washing machine using a standard amount of general laundry detergent and then spin-dried (first washing).

Based on 100 parts by weight of a total composition, 5 parts by weight of the microcapsules prepared in Examples 1 to 10 and Comparative Example 1 were added to an aqueous solution comprising 5 parts by weight of CTAB, the cotton towels were immediately treated with the resultant. Specifically, washing was performed using a fully automatic household washing machine with a water quantity of 45 L, a rinsing time of 10 minutes, and a spin-drying time of 5 minutes (second washing). Then, the cotton towels were dried for 12 hours at a humidity of 30% and a temperature of 25° C.

Twenty experienced panelists in their 20s to 40s performed sensory evaluation at three time points (immediately after washing, after drying, and after friction) to evaluate a fragrance intensity. “Immediately after washing” means the time point when the second washing was completed, “after drying” means the time point after drying in a constant temperature and humidity room for 12 hours, and “after friction” means the time point after folding the dried cotton towel horizontally twice into 4 equal parts, turning the cotton towel 90 degrees, holding the cotton towel with both hands, rubbing it gently in a circular motion with the cotton towel placed between the hands, and repeating the rubbing three times.

The fragrance intensity was graded on a scale of a minimum of 0 points to a maximum of 5 points, based on 0 points for microcapsule-free cotton towels, and this was repeated three times or more and expressed as an average value.

Table 2 below shows the results of sensory evaluation performed after preparing the composition comprising the microcapsules and washing the cotton towel.

<Evaluation Criteria>
0 points: Hardly any fragrance remains.
5 points: The fragrance strongly remains.

TABLE 2

| | Comparative | Example | Example | Example | Example | Example | Example | Example | Example | Example | Example |
|---|---|---|---|---|---|---|---|---|---|---|---|
| | Example 1 | 1 | 2 | 3 | 4 | 5 | 6 | 7 | 8 | 9 | 10 |
| Immediately | 4.12 | 2.18 | 2.11 | 3.24 | 2.18 | 2.53 | 3.11 | 2.35 | 2.15 | 2.34 | 2.53 |
| after washing | | | | | | | | | | | |
| After drying | 1.05 | 1.26 | 1.19 | 1.29 | 1.27 | 1.24 | 1.36 | 1.35 | 1.79 | 1.51 | 1.21 |
| After friction | 1.05 | 2.35 | 2.46 | 2.84 | 2.64 | 2.76 | 2.84 | 3.48 | 3.85 | 3.37 | 3.51 |

As shown in Table 2, when the microcapsules according to the present invention are used, it can be confirmed that the microcapsules have fragrance diffusion properties compared to Comparative Example 1. In particular, when a silica precursor is used as an encapsulation component, it can be confirmed that a fragrance intensity after friction was 3 or more, and the fragrance diffusion properties were high.

The reason for the excellent fragrance diffusion after drying is determined to be the result of some large microcapsule particles breaking and diffusing during a drying process.

### Examples 11 to 20. Preparation of Microcapsules According to the Type of Emulsifier

Microcapsules were prepared according to the compositions and contents of Table 3 below.

First, a continuous phase was prepared by dissolving an emulsifier in distilled water. At the same time, a dispersed phase was prepared by mixing tetraethyl orthosilicate (TEOS) and a perfume well. Then, the dispersed phase was added to the continuous phase and stirred at 8,000 rpm to prepare an emulsion, and encapsulation was performed under weakly acidic conditions.

TABLE 3

| | Example | Example | Example | Example | Example | Example | Example | Example | Example | Example | Example |
|---|---|---|---|---|---|---|---|---|---|---|---|
| | 8 | 11 | 12 | 13 | 14 | 15 | 16 | 17 | 18 | 19 | 20 |
| Tetraethyl | 10 | 10 | 10 | 10 | 10 | 10 | 10 | 10 | 10 | 10 | 10 |
| orthosilicate | | | | | | | | | | | |
| Cetyltrimonium | 0.05 | | | | | | | | | | |
| bromide (CTAB) | | | | | | | | | | | |
| Sodium lauryl | | 0.05 | | | | | | | | | |
| sulfate | | | | | | | | | | | |
| Ammonium | | | 0.05 | | | | | | | | |
| lauryl sulfate | | | | | | | | | | | |
| Cetyl alcohol | | | | 0.05 | | | | | | | |
| PEG glucoside | | | | | 0.05 | | | | | | |
| Sorbitan olivate | | | | | | 0.05 | | | | | |
| Cocamidopropyl | | | | | | | 0.05 | | | | |
| betaine | | | | | | | | | | | |
| Sodium cocoyl | | | | | | | | 0.05 | | | |
| apple amino acid | | | | | | | | | | | |
| Sodium lauroyl | | | | | | | | | 0.05 | | |
| glutamate | | | | | | | | | | | |
| Tween 20 | | | | | | | | | | 0.05 | |
| Cetyltrimonium | | | | | | | | | | | 0.05 |
| chloride | | | | | | | | | | | |
| Perfume | 10 | 10 | 10 | 10 | 10 | 10 | 10 | 10 | 10 | 10 | 10 |
| Water | To 100 | To 100 | To 100 | To 100 | To 100 | To 100 | To 100 | To 100 | To 100 | To 100 | To 100 |

### Experimental Example 2. Fragrance Intensity Evaluation after Washing

Washing evaluation was performed to confirm the residual fragrance enhancing performance of the microcapsules prepared in Examples 11 to 20.

The residual fragrance enhancement performance of these microcapsules was performed by the method of Experimental Example 1.

The sensory evaluation results are shown in Table 4 below.

<Evaluation Criteria>
0 point: Hardly any fragrance remains.
5 points: The fragrance strongly remains.

TABLE 4

| | Example | Example | Example | Example | Example | Example | Example | Example | Example | Example | Example |
|---|---|---|---|---|---|---|---|---|---|---|---|
| | 8 | 11 | 12 | 13 | 14 | 15 | 16 | 17 | 18 | 19 | 20 |
| Immediately | 2.15 | 3.85 | 3.48 | 3.26 | 2.95 | 2.97 | 2.15 | 2.35 | 2.15 | 2.85 | 2.26 |
| after washing | | | | | | | | | | | |
| After drying | 1.79 | 1.05 | 0.95 | 0.85 | 1.23 | 1.28 | 1.53 | 1.66 | 1.54 | 1.04 | 1.51 |
| After friction | 3.85 | 1.55 | 1.65 | 2.15 | 2.33 | 2.15 | 3.64 | 3.58 | 3.6 | 2.41 | 3.89 |

As shown in Table 4, when microcapsules containing perfume oil are used, it can be confirmed that the residual fragrance after friction is excellent.

In general, it can be confirmed that the microcapsules prepared using a cationic emulsifier had high residual fragrance, and the residual fragrance decreased in the order of nonionic and anionic emulsifiers. This means that when the microcapsule is prepared using a cationic emulsifier, the encapsulation occurs uniformly at the interface, so that the perfume oil retention is high and the residual fragrance after friction is high.

### Examples 21 and 22. Preparation of Surface-Modified Microcapsules

Microcapsules were prepared according to the compositions and contents of Table 5 below.

First, a continuous phase was prepared by dissolving an emulsifier in distilled water. At the same time, a dispersed phase was prepared by mixing TEOS, a silica precursor, and a perfume well. Then, the dispersed phase was added to the continuous phase and stirred at 8,000 rpm to prepare an emulsion, and encapsulation was performed under weakly acidic conditions.

TABLE 5

| | Example 8 | Example 21 | Example 22 |
|---|---|---|---|
| Tetraethyl | 10 | 10 | 10 |
| orthosilicate | | | |
| N-octyltrimethoxy | | 0.1 | |
| silicate | | | |
| Phenyltrimethoxy | | | 0.1 |
| silicate | | | |
| Cetyltrimonium | 0.05 | 0.05 | 0.05 |
| bromide (CTAB) | | | |
| Perfume | 10 | 10 | 10 |
| Water | To 100 | To 100 | To 100 |

### Experimental Example 3. Fragrance Intensity Evaluation After Washing

Washing evaluation was performed to confirm the residual fragrance enhancing performance of the microcapsules prepared in Examples 21 and 22.

The residual fragrance enhancement performance of these microcapsules was performed by the method of Experimental Example 1.

The sensory evaluation results are shown in Table 6 below.

<Evaluation Criteria>
0 point: Hardly any fragrance remains.
5 points: The fragrance strongly remains.

TABLE 6

| | Example 8 | Example 21 | Example 22 |
|---|---|---|---|
| Immediately | 2.23 | 2.18 | 2.11 |
| after washing | | | |
| After drying | 1.75 | 1.88 | 1.64 |
| After friction | 3.85 | 3.98 | 3.88 |

As shown in Table 6, it can be confirmed that the residual fragrance is maintained even when a surface of the microcapsules is modified with a functional group.

### Examples 23 to 32. Preparation of Microcapsules Sized According to Emulsifier Content

Microcapsules were prepared according to the compositions and contents of Table 7 below.

First, a continuous phase was prepared by dissolving an emulsifier in distilled water. At the same time, a dispersed phase was prepared by mixing TEOS and a perfume well. Then, the dispersed phase was added to the continuous phase and stirred at 8,000 rpm to prepare an emulsion, and encapsulation was performed under weakly acidic conditions.

In particular, in Examples 31 and 32, in order to prepare microcapsules of a smaller size, after the emulsion preparation process, tip sonicator treatment was performed for 5 minutes, and then encapsulation was performed under weakly acidic conditions.

TABLE 7

| | Example | Example | Example | Example | Example | Example | Example | Example | Example | Example | Example |
|---|---|---|---|---|---|---|---|---|---|---|---|
| | 23 | 24 | 25 | 26 | 27 | 8 | 28 | 29 | 30 | 31 | 32 |
| Tetraethyl | 10 | 10 | 10 | 10 | 10 | 10 | 10 | 10 | 10 | 10 | 10 |
| orthosilicate | | | | | | | | | | | |
| Cetyltrimonium | 0.0001 | 0.0005 | 0.001 | 0.005 | 0.01 | 0.05 | 0.1 | 0.5 | 1 | 1.5 | 2 |
| bromide (CTAB) | | | | | | | | | | | |
| Perfume | 10 | 10 | 10 | 10 | 10 | 10 | 10 | 10 | 10 | 10 | 10 |
| Water | To 100 | To 100 | To 100 | To 100 | To 100 | To 100 | To 100 | To 100 | To 100 | To 100 | To 100 |
| Size (μm) | X | 8.9 | 5.31 | 3.5 | 2.5 | 1.5 | 1.3 | 1.2 | 1.1 | 0.5 | 0.1 |

### Experimental Example 4. Microcapsule Particle Size Measurement

A particle size of the microcapsules prepared in Examples 23 to 32 was measured.

The particle size was measured using a Matersizer 3000 (Malvern, UK).

When 0.0001% of the emulsifier was used (Example 23), it could be confirmed that phase separation occurred because emulsification was not performed, but when 0.0005% or more of the emulsifier was used, emulsification began and the microcapsules were prepared.

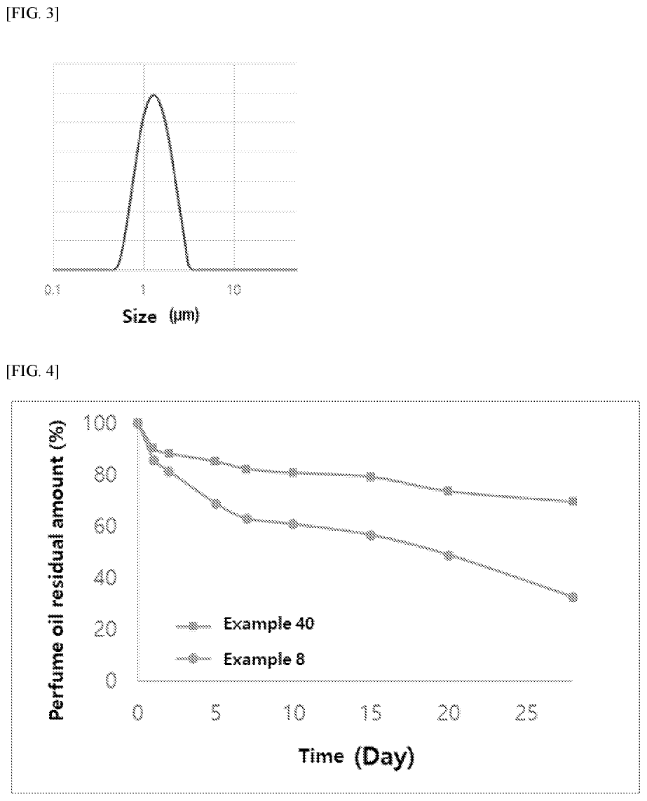

In particular, FIG. 3 is a result of measuring a particle size of the microcapsules prepared in Example 8, and it can be confirmed that the microcapsules have an average particle diameter of 1.5 μm.

### Experimental Example 5. Fragrance Intensity Evaluation After Washing

Washing evaluation was performed to confirm the residual fragrance enhancing performance of the microcapsules prepared in Examples 23 to 32.

The residual fragrance enhancement performance of these microcapsules was performed by the method of Experimental Example 1.

The sensory evaluation results are shown in Table 8 below.

<Evaluation Criteria>
0 point: Hardly any fragrance remains.
5 points: The fragrance strongly remains.

TABLE 8

| | Example | Example | Example | Example | Example | Example | Example | Example | Example | Example | Example |
|---|---|---|---|---|---|---|---|---|---|---|---|
| | 23 | 24 | 25 | 26 | 27 | 8 | 28 | 29 | 30 | 31 | 32 |
| Immediately | 4.53 | 4.35 | 3.51 | 2.35 | 2.15 | 2.23 | 2.25 | 2.05 | 2.05 | 2.21 | 2.05 |
| after washing | | | | | | | | | | | |
| After drying | 0.5 | 0.5 | 0.7 | 0.8 | 1 | 1.75 | 1.64 | 1.7 | 1.61 | 1.34 | 1.15 |
| After friction | 0.5 | 0.5 | 1.2 | 1.5 | 2.5 | 3.85 | 3.85 | 3.84 | 3.88 | 3.53 | 1.25 |

As shown in Table 8, it can be confirmed that the residual perfume increases as the size of the microcapsule decreases. It was judged that as the particle is larger, the capsule could not withstand the tension acting on the surface of the capsule and was broken, thereby failing to maintain the residual fragrance. In addition, when the emulsifier was used in an amount of 0.1% by weight or more, it could be confirmed that there was almost no change in particle size and the residual fragrance was similar.

In addition, when small-sized particles are prepared through sonicator treatment, it can be confirmed that the residual fragrance starts to decrease from 0.5 μm.

### Examples 33 to 38 and Comparative Examples 2 and 3. Preparation of Microcapsules According to Composition of Microcapsule Shell

Microcapsules were prepared according to the compositions and contents of Table 9 below.

First, a continuous phase was prepared by dissolving an emulsifier in distilled water. At the same time, a dispersed phase was prepared by mixing TEOS and a perfume well. Then, the dispersed phase was added to the continuous phase and stirred at 8,000 rpm to prepare an emulsion, and encapsulation was performed under weakly acidic conditions.

TABLE 9

| | Example | Example | Example | Example | Example | Example | Example | Comparative | Comparative |
|---|---|---|---|---|---|---|---|---|---|
| | 8 | 33 | 34 | 35 | 36 | 37 | 38 | Example 2 | Example 3 |
| Tetraethyl | 10 | 0.1 | 0.5 | 1 | 5 | 15 | 20 | 30 | 50 |
| orthosilicate | | | | | | | | | |
| Cetyltrimonium | 0.05 | 0.05 | 0.05 | 0.05 | 0.05 | 0.05 | 0.05 | 0.05 | 0.05 |
| bromide (CTAB) | | | | | | | | | |
| Perfume | 10 | 10 | 10 | 10 | 10 | 10 | 10 | 10 | 10 |
| Water | To 100 | To 100 | To 100 | To 100 | To 100 | To 100 | To 100 | To 100 | To 100 |
| Condition | Good | Phase | Good | Good | Good | Good | Good | Gelation | Gelation |
| after 28 | | separation | | | | | | start | |
| days at room | | | | | | | | | |
| temperature | | | | | | | | | |

As a result of checking the change at room temperature for 28 days depending on a TEOS content, it could be confirmed that when 0.1% by weight of TEOS was used, phase separation occurred because capsules were not properly made, when 30% by weight or more of TEOS was used, gelation started, and when 50% by weight or more of TEOS was used, the problem of gel formation had occurred.

### Examples 39 to 43. Preparation of Double-Walled Microcapsules

Microcapsules were prepared according to the compositions and contents of Table 10 below.

First, a continuous phase was prepared by dissolving an emulsifier in distilled water. At the same time, a dispersed phase was prepared by mixing each of TEOS and a perfume well. Then, the dispersed phase was added to the continuous phase and stirred at 8,000 rpm to prepare an emulsion, and encapsulation was performed under weakly acidic conditions. Thereafter, 1 to 9% of TEOS was added, and then stirred at 400 rpm to perform additional encapsulation.

TABLE 10

| | Example | Example | Example | Example | Example | Example |
|---|---|---|---|---|---|---|
| | 39 | 40 | 41 | 42 | 43 | 8 |
| Tetraethyl | 10 | 10 | 10 | 10 | 10 | 10 |
| orthosilicate | | | | | | |
| Tetraethyl | 1 | 3 | 5 | 7 | 9 | 0 |
| orthosilicate | | | | | | |
| (addition) | | | | | | |
| Cetyltrimonium | 0.0001 | 0.0005 | 0.001 | 0.005 | 0.01 | 0.05 |
| bromide (CTAB) | | | | | | |
| Perfume | 10 | 10 | 10 | 10 | 10 | 10 |
| Water | To 100 | To 100 | To 100 | To 100 | To 100 | To 100 |

### Experimental Example 6. Fragrance Intensity Evaluation After Washing

Washing evaluation was performed to confirm the residual fragrance enhancing performance of the microcapsules prepared in Examples 39 to 43. Before washing, the microcapsules were put in a 5% CTAB solution and stored at 50° C. for 1 month.

The residual fragrance enhancement performance of these microcapsules was performed by the method of Experimental Example 1. The sensory evaluation results are shown in Table 11 below.

<Evaluation Criteria>
0 point: Hardly any fragrance remains.
5 points: The fragrance strongly remains.

TABLE 11

| | Example | Example | Example | Example | Example | Example |
|---|---|---|---|---|---|---|
| | 39 | 40 | 41 | 42 | 43 | 8 |
| Immediately | 2.23 | 2.25 | 2.05 | 2.05 | 2.21 | 2.2 |
| after washing | | | | | | |
| After drying | 1.2 | 1.23 | 1.3 | 1.25 | 1.3 | 1.2 |
| After friction | 2.62 | 3.15 | 3.12 | 3.25 | 3.24 | 2.45 |

As shown in Table 11, as a content of silica contained in a microcapsule shell increases, it can be confirmed that the microcapsule maintains its fragrance even in a harsh environment (environment containing a high content of surfactant at high temperature). It was judged that this is the result of the thickening of the capsule itself.

FIG. 2D shows a SEM picture of the microcapsules prepared in Example 40, it can be confirmed that the surface is smooth and the thickness also increased from 22 nm to 38 nm compared to FIG. 2C corresponding to Example 8.

### Experimental Example 7. Microcapsule Evaluation

In order to check whether the outer wall (shell) of the microcapsule was properly prepared, the microcapsule prepared in Example 8 was put into ethanol and the perfume was extracted for a sufficient time, and then centrifugation was performed using a centrifuge. Thereafter, water was added, dispersed, and lyophilized to obtain silica particles in the form of fine powder, and a microcapsule shape was confirmed using scanning electron microscope.

FIG. 2 shows the results of confirming the shape of the microcapsule. As shown in FIGS. 2A and 2B, it can be confirmed that the outer wall of the mineral material is formed on the surface of the microcapsule.

Meanwhile, in order to check the stability of the microcapsules, 5 parts by weight of each of the microcapsules prepared in Examples 8 and 40 was added to an aqueous solution comprising 5 parts by weight of CTAB based on 100 parts by weight of a total composition, and then placed in a chamber at 50° C. and stored for 4 weeks. Samples were taken on days 1, 2, 5, 7, 10, 14, 20, and 28 to filter out the microcapsules using a 0.45 μm syringe filter and put in ethanol, and an actual concentration of the samples was measured by measuring the absorbance of the perfume remaining inside the microcapsule using a sonicator using a UV-Vis spectrophotometer.

FIG. 4 shows the results of confirming the stability of the microcapsules.

As shown in FIG. 4, in the case of Example 40, it can be confirmed that the perfume was maintained at about 80% for 4 weeks even at 50° C. Through this, it can be indirectly confirmed that the microcapsules can maintain the perfume for a long time even under harsh conditions containing a large amount of a surfactant.

### Examples 44 to 55. Preparation of Microcapsules According to pH

Microcapsules were prepared according to the compositions and contents of Table 12 below. First, a continuous phase was prepared by dissolving an emulsifier in distilled water. At the same time, a dispersed phase was prepared by mixing an encapsulation component and a perfume well. The dispersed phase was added to the continuous phase and stirred at 8,000 rpm to prepare an emulsion.

Thereafter, the pH of the emulsion was adjusted as shown in Table 12 using 1.0 N HCl and 1.0 N NaOH, and then encapsulation was performed.

TABLE 12

| | Example | Example | Example | Example | Example | Example | Example |
|---|---|---|---|---|---|---|---|
| | 44 | 45 | 46 | 47 | 40 | 48 | 49 |
| Tetraethyl | 10 | 10 | 10 | 10 | 10 | 10 | 10 |
| orthosilicate | | | | | | | |
| Tetraethyl | 3 | 3 | 3 | 3 | 3 | 3 | 3 |
| orthosilicate | | | | | | | |
| (addition) | | | | | | | |
| Perfume | 10 | 10 | 10 | 10 | 10 | 10 | 10 |
| Cetyltrimonium | 0.05 | 0.05 | 0.05 | 0.05 | 0.05 | 0.05 | 0.05 |
| bromide (CTAB) | | | | | | | |
| Water | To 100 | To 100 | To 100 | To 100 | To 100 | To 100 | To 100 |
| pH | 1 | 1.5 | 2 | 2.5 | 3 | 3.5 | 4 |
| Condition | Phase | Phase | Emulsi- | Emulsi- | Emulsi- | Emulsi- | Emulsi- |
| | separation | separation | fication | fication | fication | fication | fication |

| | Example | Example | Example | Example | Example | Example |
|---|---|---|---|---|---|---|
| | 50 | 51 | 52 | 53 | 54 | 55 |
| Tetraethyl | 10 | 10 | 10 | 10 | 10 | 10 |
| orthosilicate | | | | | | |
| Tetraethyl | 3 | 3 | 3 | 3 | 3 | 3 |
| orthosilicate | | | | | | |
| (addition) | | | | | | |
| Perfume | 10 | 10 | 10 | 10 | 10 | 10 |
| Cetyltrimonium | 0.05 | 0.05 | 0.05 | 0.05 | 0.05 | 0.05 |
| bromide (CTAB) | | | | | | |
| Water | To 100 | To 100 | To 100 | To 100 | To 100 | To 100 |
| pH | 4.5 | 5 | 5.5 | 6 | 7 | 8 |
| Condition | Emulsi- | Emulsi- | Emulsi- | Phase | Phase | Phase |
| | fication | fication | fication | separation | separation | separation |

As a result of preparing the microcapsules, when the pH was less than 2 and when the pH was 6 or more, phase separation occurred without microcapsules forming during a preparation process. At pH 2 to 5.5, it was confirmed that an emulsified state was maintained even after the encapsulation was performed.

### Experimental Example 8. Fragrance Intensity Evaluation After Washing

Washing evaluation was performed to confirm the residual fragrance enhancing performance of the capsule according to a pH of the microcapsules prepared in Examples 46 to 52. Before washing, the microcapsules were put in a 5% CTAB solution and stored at 50° C. for 1 month.

The residual fragrance enhancement performance of these microcapsules was performed by the method of Experimental Example 1. The sensory evaluation results are shown in Table 13 below.

<Evaluation Criteria>
0 point: Hardly any fragrance remains.
5 points: The fragrance strongly remains.

TABLE 13

| | Example | Example | Example | Example | Example | Example | Example | Example |
|---|---|---|---|---|---|---|---|---|
| | 46 | 47 | 40 | 48 | 49 | 50 | 51 | 52 |
| Immediately | 2.31 | 2.21 | 2.25 | 2.31 | 2.25 | 2.45 | 2.58 | 2.66 |
| after washing | | | | | | | | |
| After drying | 1.10 | 1.15 | 1.23 | 1.31 | 1.25 | 1.15 | 1.11 | 0.98 |
| After friction | 3.12 | 3.28 | 3.15 | 3.21 | 3.11 | 2.45 | 2.15 | 1.55 |

As shown in Table 13, it can be confirmed that the microcapsules prepared at a pH of 2 to 4 have high fragrance retention.

This means that in order to manufacture microcapsules with fewer defects, it is necessary to prepare microcapsules in an appropriate pH environment, and the microcapsules that can preserve an active ingredient for a long time can be prepared only when the microcapsules are prepared at a pH of 2 to 4.

### INDUSTRIAL APPLICABILITY

In the present invention, the active ingredient can be stably loaded by using the mineral materials, and since microcapsules prepared by the preparation method according to the present invention have the same encapsulation components as soil components, there is no environmental problem when the encapsulation component is discharged into nature, and thus microplastic issues can be avoided. In addition, since the microcapsules prepared by the preparation method according to the present invention have an outer wall of a capsule having a dense structure, elution of the active ingredient can be prevented.

1. A method of preparing microcapsules, comprising:
preparing an emulsion by mixing a continuous phase containing an emulsifier and a dispersed phase containing an encapsulation component and an active ingredient; and
encapsulating the emulsion,
wherein the emulsifier comprises one or more selected from the group consisting of an anionic surfactant, a cationic surfactant, an amphoteric surfactant, and a nonionic surfactant, and
the encapsulation component comprises one or more selected from the group consisting of a silica precursor, a titanium oxide precursor, and a zirconium oxide precursor.

2. The method of claim 1, wherein the emulsifier is the cationic surfactant.

3. The method of claim 1, wherein the emulsifier is cetyltrimethylammonium bromide (CTAB) or cetyltrimethylammonium chloride (CTAC).

4. The method of claim 1, wherein a content of the emulsifier is 0.00001 to 10 parts by weight based on a total weight of the emulsion.

5. The method of claim 1, wherein the encapsulation component comprises one or more selected from the group consisting of compounds represented by Chemical Formulas 1 to 4 below: 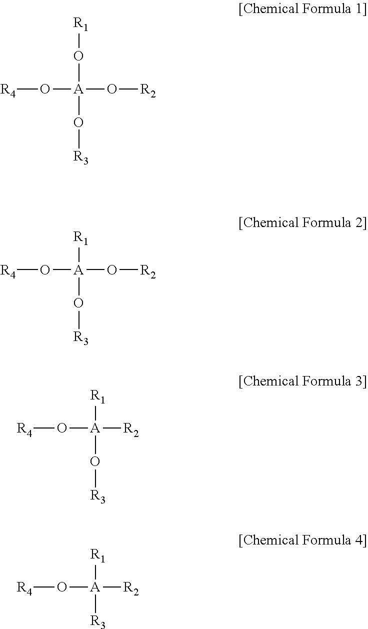
In Chemical Formulas 1 to 4, A is silicon, titanium, or zirconium,
R₁ to R₄ are each independently hydrogen or a C1 to C8 alkyl group with a functional group substituted or unsubstituted at the terminal, and
the functional group comprises amine, hydroxy, amide, carboxy, vinyl, epoxy, phenyl, or mercapto.

6. The method of claim 1, wherein the silica precursor comprises one or more selected from the group consisting of tetramethyl orthosilicate, tetraethyl orthosilicate, tetrapropyl orthosilicate, tetrabutyl orthosilicate, methyltrimethoxysilicate, trimethylethoxysilicate, butyltrimethoxysilicate, N-propyltrimethoxysilane, N-octyltrimethoxysilane, aminopropyltrimethoxysilicate, phenyltrimethoxysilane, and 3-mercaptopropyltrimethoxysilane.
the titanium oxide precursor comprises one or more selected from the group consisting of titanium methoxide, titanium ethoxide, and titanium butoxide, and
the zirconium oxide precursor comprises one or more selected from the group consisting of zirconium methoxide, zirconium ethoxide, and zirconium butoxide.

7. The method of claim 1, wherein a content of the encapsulation component is 0.001 to 30 parts by weight based on a total weight of the emulsion.

8. The method of claim 1, wherein the active ingredient comprises one or more selected from the group consisting of a perfume, a sunscreen, a dye, a catalyst, an antioxidant, and a drug.

9. The method of claim 1, wherein a content of the encapsulation component is 0.5 to 3 times that of the active ingredient.

10. The method of claim 1, wherein a content of the encapsulation component and the active ingredient is 35 parts by weight or less based on a total weight of the emulsion.

11. The method of claim 1, wherein the encapsulation is performed at a pH of 2 to 5.

12. Microcapsules prepared by the method of preparing microcapsules of claim 1 and having a particle size of 0.1 to 1,000 μm.

13. A laundry product comprising the microcapsules of claim 12.

14. (canceled)
